(19) United States
(12) Patent Application Publication
(10) Pub. No.: US 2002/0136700 A1
(43) Pub. Date: Sep. 26, 2002

(54) Vehicle and concentrates for customized personal care products

(75) Inventors: Marion Louise Margosiak, Hamden, CT (US); Christy A. Bridges, Fairfield, CT (US); Marcina Siciliano, New Haven, CT (US); Rosa Paredes, Shelton, CT (US); Brian John Dobkowski, Milford, CT (US); Craig Stephen Slavtcheff, Guilford, CT (US)

Correspondence Address: UNILEVER, PATENT DEPARTMENT, 45 RIVER ROAD, EDGEWATER, NJ, 07020, US

(73) Assignee: Unilever Home & Personal Care USA,Division of Conopco, Inc.

(21) Appl. No.: 09/930,622

(22) Filed: Aug. 15, 2001

Related U.S. Application Data
(60) Provisional application No. 60/227,886, filed on Aug. 25, 2000.

(51) Int. Cl. A61K 7/075

(52) U.S. Cl. 424/70.21

(57) ABSTRACT

A vehicle, and a fragrance concentrate are described for providing a customized, personal care product to a consumer at a location, typically the point of sale, that is remote from a second location in which a personal care product base composition is prepared. The custom personal care product typically includes a product base, a fragrance concentrate, and one or more performance agent concentrates that may contain benefit agents, botanical extracts, active ingredients for skin and hair, and the like. The inventive vehicle is substituted to the extent that an insufficient amount of performance agents are selected by the consumer to obtain optimum, final product, component concentrations.

### PRIORITY

This application claims priority to provisional application No. 60/227,886 filed Aug. 25, 2000.

### BACKGROUND OF THE INVENTION

Many personal care products such as body wash, facial cleansing gel, hair shampoo, hair conditioner, face lotion and other personal care products that are currently available are provided as prepared formulations. A drawback of such products is that the user cannot alter the formulation to accommodate their particular skin and hair characteristics, personal preferences, or to provide specialized treatment. A further drawback is the uncertainty of the age and freshness of the prepared formulation which may have been prepared many months or years before the product is sold.

Multiple-part product kits that contain separate components of e.g. a hair shampoo, hair conditioner, cosmetics, or other personal care products are available from many professional salons and some retail personal care outlets. However, the user is still unable to customize the individual formulations in light of the consumer's unique personal care preferences and requirements.

Therefore, one aspect of the invention is to provide a vehicle and various performance agent concentrates that are compatible with a wide range of personal care product bases and that enable a user to formulate a variety of personal care product compositions to best suit the needs of the consumer. Compatibility is herein defined as the ability of the final product formulated with the inventive components to be stable. Stability is herein defined as the maintenance of each of the following product characteristics: prevention of noticeable phase separation or precipitation, no change in viscosity beyond about 25%, preferably about 3%, and most preferably about 1% at 25 C. and no signicant change in performance properties such as product foaming ability, texture, clarity (if the product was transparent or translucent initially after blending), and skin feel. Significant change is herein defined as greater than about 25%, preferably greater than about 10% and most preferably greater than about 5% of the performance property in question as measured by art recognized techniques.

Another aspect of the invention is to provide a vehicle and performance agent and fragrance concentrates that are easy to use such that the user may be a lay-person who is able formulate a customized personal care product. A vehicle is herein defined as a composition compatible with a personal care product base, performance agent concentrates, and fragrance concentrates, and which contains less than 0.5% of a performance agent and a fragrance agent, preferably less than 0.1%, and most preferably contains substantially no performance and fragrance agent. Performance agents are herein defined as any i5 colorant or benefit agent comprising any of the following: botanical extracts, sunscreen agents, emollients, vegetable oils, protein, honey, vitamins, or active agents to prevent or treat undesired skin and hair conditions, or blends thereof, and the like.

### SUMMARY OF THE INVENTION

A vehicle and at least one performance or fragrance agent concentrate are provided for preparing a customized, personal care product for a consumer at a location remote from a second location in which a personal care product base composition is prepared. Typically an assortment of product bases of various personal care products and an assortment of inventive variants of performance agent and fragrance concentrates are provided for custom blending. The product bases and inventive performance agent and fragrance concentrates are compatible with each other when all the components are mixed together forming a stable personal care finished product. The consumer typically selects, in any sequence, one personal care base composition and at least one variant from separate concentrates of performance agents, and fragrances. The user doses, in any sequence, the consumer selected personal care base composition, performance agents, fragrance concentrates, and, if necessary, sufficient vehicle into a container, mixes the contents until uniform, and labels the container.

A sufficient quantity of an inventive vehicle composition may be dosed in the container if an insufficient number of performance agents and fragrance concentrates are selected by the consumer. A sufficient amount is herein defined as that quantity necessary for maintaining the optimum concentration and stability of product base ingredients had a sufficient number of performance agents and fragrance concentrates been selected to begin with. The ability to vary both the number and identity of performance agent selections serves to provide many choices for the consumer in customizing the product.

The inventive vehicle, performance agent, and fragrance concentrates may be used for formulation with various cleansing and conditioning personal care product base compositions such as a body wash, body lotion, body mist spray, hydroalcoholic toner, facial cleansing gel, hand cleanser, hair shampoo, hair conditioner, face lotion, deodorant, bar soap, a bath foam, bath salts, and the like.

### DETAILED DESCRIPTION OF THE INVENTION

In one aspect of the invention is a personal care product vehicle composition suitable for preparing stable personal care compositions, and performance agent concentrates thereof, comprising:

from about 5 to 99 w/w % of at least one solvent;

from about 0 to 40 w/w % of at least one solubilizing agent;

from about 0.01 to 10 w/w % of a preservative; and wherein said vehicle contains less than about 0.5 w/w % of a fragrance and a performance agent.

The relative percentage increase in viscosity of said personal care product doesn't exceed about 3%, preferably about 1%, after the addition of the inventive vehicle in substitution for at least one component selected from said fragrance and said performance agent. By substitution is meant where the consumer does not select the maximum number of performance agents and maximum intensity or amount of fragrance designed for formulating a particular product, and the inventive vehicle is therefore substituted to the extent necessary to maintain the optimum concentration of the product base, fragrance and performance agent ingredients actually selected.

Preferably the inventive vehicle composition contains one or more solvents in an amount of at least about 90 w/w %; and one or more solubilizing agents in a total amount below about 10 w/w %, the solubilizing agents being selected from polyethylene glycol ether of a fatty alcohol, a polyethylene glycol ether of hydrogenated castor oil, a polyethylene glycol derivative of a sorbitan ester, propylene glycol, a polysorbate, a glycerol ester, a polyethylene glycol derivative of a glycerol ester, an alkyl phosphate, and an alkyl sulfate, and the like; and a preservative in an amount below about 5.0 w/w %. More preferably the solubilizing agent is selected from peg-40 hydrogenated castor oil, trideceth-9, or any mixture thereof. Most preferably the solubilizing agent is selected from a blend of peg-40 hydrogenated castor oil and trideceth-9 obtainable as Solubilizer 2/014160 from Dragoco Inc. (Totowa, N.J.). More preferably the inventive vehicle contains less than 0.1% of a performance agent and a fragrance, most preferably the vehicle contains substantially no performance agent and fragrance.

Preferably the inventive vehicle composition contains at least one solvent selected from water, monohydric and polyhydric alcohols, or a blend thereof. Among suitable monohydric alcohols are ethanol, isopropanol, butanol, hexanol and combinations thereof, and the like. Most preferred is ethanol. Suitable polyhydric alcohols include glycerin (known also as glycerol), propylene glycol, dipropylene glycol, polyethylene glycol, sorbitol, hydroxypropyl sorbitol, hexylene glycol, 1,3-butylene glycol, isopropylene glycol and mixtures thereof, and the like. Most preferred are propylene glycol and polyethylene glycol.

Enhancement of a visual effect of the inventive vehicle and fragrance concentrates may be achieved by employing a colorant. By the term colorants is meant any water-soluble or dispersible dye, which imparts a color in the visible range to that of the composition. Colors may include red, yellow, blue, green as well as shades therebetween. Examples of useful dyes include the following FD&C colorants; Red no. 4, Blue no. 1, Blue no. 2, Yellow no. 5, Yellow no. 6, and the following D&C colorants Yellow no. 10, Green no. 5, Red no. 33, Red no. 22, Red no. 28, Green no. 8, Orange no. 4, Ext. Violet no. 2, Brown no. 1 Replacement, Herc Jetine Black, and the like. Useful concentration levels of these colorants may range from 0.001 to about 30%, preferably from 0.01 to 0.5% by weight.

Colorant stability may be a problem in certain compositions of the present invention. This is especially so since storage containers and consumer packaging are preferably transparent, or at least translucent, which allows a consumer to distinguish a personal care product from other products. Sunlight and ambient light can over time destroy the colorant. For these purposes, it has been found useful to incorporate an UV protectant, particularly a substance absorbing ultraviolet radiation within the range of 290 to 400 nm. Useful stabilizers may be selected from the para-amino benzoates, salicylates, cinnamates, benzophenones, anthralinates, azoles and digalloyl functional groups. Levels of the color stabilizing agents may range from about 0.001 to about 5%, preferably from about 0.001 to about 0.5%, and more preferably between about 0.1 and 0.5% by weight. The colorant and the color-stabilizing agent are preferably water-soluble materials. Especially useful as the color stabilizing agent are benzophenone-2 and benzophenone-4 (CTFA nomenclature) available as Uvinul D-50 and Uvinul MS-40 respectively from BASF Corporation (Washington N.J.), octylmethoxy cinnamate and urocanic acid.

Preservatives are desirably incorporated into the compositions of this invention to protect against the growth of potentially harmful microorganisms. Suitable preservatives for the compositions of this invention include alkyl esters of para-hydroxybenzoic acid, hydantoin derivatives, propionate salts, quaternary ammonium compounds, and the like. Useful preservatives are selected to satisfy the preservative challenge test and to provide product stability. Preferred preservatives include DMDM Hydantoin, lodopropynyl Butylcarbamate, polyaminocarboxylic acid chelates and salts thereof, and phosphonate chelates and salts thereof, and the like. The preservatives should be selected having regard for the use of the composition and possible incompatibilities between the preservatives and other ingredients in the personal care products. Preservatives are preferably employed in amounts ranging from about 0.01% to about 2% by weight of the composition.

More preferably the inventive vehicle for optional use as a substituent for a portion of fragrance concentrate contains an UV inhibitor type preservative and substantially no solubilizing agent.

The inventive vehicle composition remains stable as defined above after being blended with at least one fragrance in a concentration of about 0.01 to 10.0 w/w %, preferably about 0.05 to 5.0 w/w %; with at least one performance agent in a concentration of about 0.01 to 10.0 w/w %, preferably about 1.0 to 5.0 w/w %; and with at least one personal care composition base in a concentration of about 10.0 to 95.0 w/w %, preferably about 80.0 to 95.0 w/w %.

Preferably performance agents are selected from botanical extracts, vegetable oils, protein, honey and vitamins such as cucumber extract, ginseng extract, wheat protein, cactus extract, rosemary extract, honey extract, glycerin, fruit acid extract, rose hips extract, grape seed extract, aloe vera gel, tea tree extract, evening primrose extract, sea kelp extract, ginkgo biloba extract, chamomile extract, mallow extract, silk protein, echinacea extract, sage extract, panthenol, vitamin e acetate, vitamin a palmitate, borage seed oil, and sunflower seed oil, or blends thereof, and the like.

In another aspect of the invention is a fragrance concentrate comprising:

from about 5 to about 80 w/w % of a fragrance composition;

from about 0.001 to about 30 w/w %; preferably about 0.01 to about 0.5 w/w % of a colorant;

from about 5 to about 80 w/w % of a solvent; and

from about 0.01 to 10 w/w % of a preservative.

Preferably, the fragrance concentrate contains a solvent and a preservative as described above. Preferably the preservative is present in a concentration range of 0.01 to 5.0 w/w %. Preferably the fragrance concentrate also contains an UV inhibitor type preservative as described above.

The personal care product base compositions useful in the invention typically contain one or more of the following: a solvent such as water, monohydric and/or polyhydric alcohols, and the like; soaps, surfactants such as anionic, cationic, amphoteric, zwitterionc surfactants, and the like; conditioning agents such as cationic polymers, silicone polymers and the like; thickening agents such as acrylates, polysaccharide polymers, and the like; lathering aids such as alkanolamides and the like; emollients such as vegetable oils, fatty esters, and the like; pH adjusters, and preservatives. Other examples of useful solvents, surfactants, conditioning agents, thickening agents, lathering aids, emollients, pH adjusters, and preservatives are listed in the CTFA Cosmetic Ingredient Handbook, second edition, 1990; which is here incorporated by reference.

Other examples of useful anionic, amphoteric, and zwitterionic surfactants, and foam boosters of the personal care product base compositions useful in the invention are described in U.S. Pat. No. 5,221,530 issued to Janchitraponvej, et. al. on Jun. 22, 1993; incorporated herein by reference. Examples of useful hair shampoo, conditioner, and styling base compositions useful in the invention are described in U.S. Pat. No. 5,993,792 issued to Rath, et. al. on Nov. 30, 1999 incorporated herein by reference. Preferably the personal care product base has a viscosity in the range of 1000 to 100,000 cps at 25 C., more preferably in the range of 2,000 to 30,000 cps at 25 C.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated.

### EXAMPLE 1

The custom formulation of separate embodiments of an inventive body lotion, hair conditioner, facial cleanser, body mist, toner, body wash, instant hand cleanser, hair shampoo and face lotion are illustrated in Table 1A. Specifically illustrated is the choice of a single base formula corresponding to the product type above, the choice of one of three different embodiments of inventive fragrance concentrates, or alternatively, of a single, inventive fragrance concentrate, to be dispensed either in one, two or three portions in order to provide moderate, strong, and stronger fragrance intensity levels, and the choice of one or more of four different embodiments of inventive benefit agents. Tables 1B and 1C illustrate embodiments of the inventive fragrance vehicle or blank and the inventive benefit vehicle or blank respectively.

### EXAMPLE 2

Table 2 shows in further detail the composition of the embodiment of the custom body lotion product base depicted in table 1. Table 2a shows the composition of a body lotion base concentrate, tables 2b and 2c show the composition of embodiments of inventive fragrance and benefit agent concentrates respectively that the consumer may select, and table 2d shows how the consumer selected agents are blended together to make the custom body lotion product. If the consumer does not pick sufficient choices of a fragrance, benefit agent, or other performance agent; sufficient inventive vehicle or blank fragrance/benefit agents, as depicted in tables 2e and 2f respectively, are substituted. The effect on the viscosity of the body lotion product (measured at 25 C., Brookfield RV6@20 rpm, 60s) is shown in table 2g when varying quantities of fragrance concentrate are used for three cases: 1) when the inventive vehicle is substituted for specified levels of fragrance concentrate, 2) when additional product base is substituted for specified levels of fragrance concentrate, or 3) when neither vehicle or product base substituted for specified levels of fragrance concentrate.

### EXAMPLE 3

Table 3a shows in further detail the composition of an embodiment of the custom hair conditioner product base depicted in table 1. Inventive fragrance and benefit agents are depicted in tables 2b and 2c above. Table 3b shows how the consumer selected agents are blended together to make the custom hair conditioner product. As for the body lotion in Example 2, if the consumer does not pick sufficient choices for a fragrance or benefit agent for the hair conditioner, sufficient inventive vehicle or blank fragrance/benefit agents as depicted in tables 2e and 2f respectively are substituted.

TABLE 1A

| | | | | | Benefit #1 | Benefit #2 | Benefit #3 | Benefit #4 |
|---|---|---|---|---|---|---|---|---|
| | | | | | Solvent | Solvent | Solvent | Solvent |
| | | | | | (Water) | (Water) | (Water) | (Water) |
| | | Fragrance | Fragrance | Fragrance | Solubilizing | Solubilizing | Solubilizing | Solubilizing |
| | | Allotment #1 | Allotment #2 | Allotment #3 | Agent (PEG- | Agent (PEG- | Agent (PEG- | Agent (PEG- |
| | | Solvent | Solvent | Solvent | 40 | 40 | 40 | 40 |
| | | (Propylene | (Propylene | (Propylene | Hydrogenated | Hydrogenated | Hydrogenated | Hydrogenated |
| | | Glycol) | Glycol) | Glycol) | Castor Oil & | Castor Oil & | Castor Oil & | Castor Oil & |
| | | Preservatives | Preservatives | Preservatives | Trideceth-9) | Trideceth-9) | Trideceth-9) | Trideceth-9) |
| | | (Glydant | (Glydant | (Glydant | Preservatives | Preservatives | Preservatives | Preservatives |
| | | Plus & Edta) | Plus & Edta) | Plus & Edta) | (Glydant | (Glydant | (Glydant | (Glydant |
| | | UV-Inhibitor | UV-Inhibitor | UV-Inhibitor | Plus & Edta) | Plus & Edta) | Plus & Edta) | Plus & Edta) |
| | | (Benzophe- | (Benzophe- | (Benzophe- | Performance | Performance | Performance | Performance |
| Product | Base | none-2) Color | none-2) Color | none-2) Color | Agent | Agent | Agent | Agent |
| Form | Formula | & Fragrance | & Fragrance | & Fragrance | (Benefit) | (Benefit) | (Benefit) | (Benefit) |
| Body | 90.435 | .87% | .87% | .87% | 1.74% | 1.74% | 1.74% | 1.74% |
| Lotion | % | | | | | | | |
| Hair | 91.478 | .522% | .522% | .522% | 1.74% | 1.74% | 1.74% | 1.74% |
| Conditioner | % | | | | | | | |
| Facial | 92.2636 | .26% | .26% | .26% | 1.74% | 1.74% | 1.74% | 1.74% |
| Cleanser | % | | | | | | | |
| Body Mist | 86% | 2% | 2% | 2% | 2% | 2% | 2% | 2% |
| Toner | 91.1% | .3% | .3% | .3% | 2% | 2% | 2% | 2% |
| Body | 89.5% | 1.33% | 1.33% | 1.33% | 1.625% | 1.625% | 1.625% | 1.625% |
| Wash | | | | | | | | |
| Instant | 83.49% | .56% | .56% | .56% | 3.71% | 3.71% | 3.71% | 3.71% |
| Hand | | | | | | | | |
| Cleanser | | | | | | | | |
| Hair | 90.8% | .84% | .84% | .84% | 1.675% | 1.675% | 1.675% | 1.675% |
| Shampoo | | | | | | | | |
| Face | 93.03% | .084% | .084% | .084% | 1.68% | 1.68% | 1.68% | 1.68% |
| Lotion | | | | | | | | |

TABLE 1B

Fragrance Blank
Solvents (Dipropylene Glycol &
Propylene Glycol)
Preservatives (Glydant Plus &
Tetrasodium Edta)
UV-Inhibitor (Benzophenone-2)
NO COLOR

TABLE 1C

Benefit Blank
Solvent (Water)
Solubilizing Agent
(PEG-40 hydrogenated
Castor Oil and Trideceith-9)
Preservatives (Glydant Plus
and Disodium EDTA

Tables 2a-g

TABLE 2a
Base Hand Lotion formula

| COMPONENT | % wt/wt |
|---|---|
| WATER | 75-80% |
| Na2 EDTA | .04-.08 |
| magnesium aluminum silicate | .02-.04 |
| tio2 | .08-.12 |
| carbopol | 8-12 |
| Triethanolamine | 1-1.5 |
| STEARIC ACID | 2-3 |
| Cetyl Alcohol | 2-3 |
| Mineral Oil | 2-3 |
| PEG-100 Stearate | .2-1.0 |
| steramide | .2-1.0 |
| Glyceryl Stearate | .2-1.0 |
| silicone | .2-1.0 |
| C12-15 Alkyl Ethylhexanoate | .2-1.0 |
| Glydant Plus | .1-.5 |

TABLE 2d
Finished Hand Lotion Formula

| Ingredient | % wt/wt |
|---|---|
| Base Lotion Formula | 90.4% |
| Fragrance Formula | 2.6% |
| Benefits Formulas | 7.0% |

TABLE 2b
Custom fragrance formula

| Ingredient | Function | % wt/wt |
|---|---|---|
| Fragrance | Performance | 40-60% |
| | Agent | |
| Propylene Glycol | Solvent | 40-60% |
| Glydant Plus liquid | Preservative | 0.1-0.5% |
| Tetrasodium EDTA | Preservative | 0.01-0.2% |
| Benzophenone-2 | UV-Inhibitor | 0.1-0.5% |
| OPTIONAL COLORS | Performance | 0.01-3.0% |
| — | Agent | |

TABLE 2f
Custom fragrance blank formula

| Ingredient | Function | % wt/wt |
|---|---|---|
| Dipropylene Glycol | Solvent | 40-60% |
| Propylene Glycol | Solvent | 40-60% |
| Glydant Plus liquid | Preservative | 0.1-0.5% |
| Tetrasodium EDTA | Preservative | 0.01-0.2% |
| Benzophenone-2 | UV-Inhibitor | 0.1-0.5% |

TABLE 2c
Custom benefit formula

| Ingredient | Function | % wt/wt |
|---|---|---|
| DI Water | Solvent | 93-97 |
| Benefit Component | Performance Agent | 0.3-1.0% |
| Solubilizer | Solubilizing Agent | 2-6% |
| Glydant Plus liquid | Preservative | 0.1-0.5% |
| Disodium EDTA | Preservative | 0.01-0.1% |

TABLE 2e
Custom benefit blank formula

| Ingredient | Function | % wt/wt |
|---|---|---|
| DI Water | Solvent | 93-97 |
| Solubilizer | Solubilizing Agent | 2-6% |
| Glydant Plus liquid | Preservative | 0.1-0.5% |
| Disodium EDTA | Preservative | 0.01-0.1% |

TABLE 2g
Viscosity variation with and without vehicle addition

| | | | Without Vehicle | Without Vehicle |
|---|---|---|---|---|
| Fragrance amount | | With Vehicle | With Base filler | Without Base filler |
| 6 g | Viscosity | 8,700 cPs | | |
| | | 208 g Base/0 g Vehicle | | |
| | total | 230 g | | |
| 4 g | Viscosity | 8,600 cPs | 8,550 cPs | 8,700 cPs |
| | | 208 g Base/2 g Vehicle | 210 g Base/0 g Vehicle | 208 g Base/0 g Vehicle |
| | total | 230 g | 230 g | 228 g |
| 2 g | Viscosity | 8,650 cPs | 9,300 cPs | 9,050 cPs |
| | | 208 g Base/4 g Vehicle | 212 g Base/0 g Vehicle | 208 g Base/0 g Vehicle |
| | total | 230 g | 230 g | 226 g |
| 0 g | Viscosity | 8,650 cPs | 9,700 cPs | 9,650 cPs |
| | | 208 g Base/6 g Vehicle | 214 g Base/0 g Vehicle | 208 g Base/0 g Vehicle |
| | total | 230 g | 230 g | 224 g |

TABLE 3a
Base Hair Conditioner formulation

| Ingredients | % wt/wt |
|---|---|
| DI Water | 80-90 |
| Citric Acid | 0.1-0.4 |
| Hydroxyethylcellulose | 0.1-0.4 |
| Stearamidopropyl Dimethylamine | 0.2-1.0 |
| Stearyl Octyldimonium Methosulfate | 1.0-2.0 |
| BES & Stearyl Alcohol | 0.2-1.0 |
| Cetyl Alcohol | 1.5-4.0 |
| Stearyl Alcohol | 1.0-2.0 |
| Disodium EDTA | 0.1-0.4 |
| Potassium Hydroxide | 0.01-0.1 |
| Glydant Plus Liquid | 0.1-0.4 |
| dimethyl siloxane derivative & TEA- | 0.7-2.0 |
| Dodecylbenzenesulfonate | |
| Siloxane derivative | 0.7-2.0 |

TABLE 3b
Finished Hair Conditioner Product

| Ingredient | % wt/wt |
|---|---|
| Base Conditioner Formula | 91.5% |
| Fragrance Formula | 1.5% |
| Benefits Formulas | 7.0% |

The foregoing description and examples illustrate selected embodiments of the present invention. In light thereof variations and modifications will be suggested to one skilled in the art, all of which are within the scope and spirit of this invention.

We claim:

1. A personal care product vehicle composition suitable for preparing stable personal care compositions and concentrates thereof, comprising:
from about 5 to 99 w/w % of at least one solvent;
from about 0 to 60 w/w % of at least one solubilizing agent;
from about 0.01 to 10 w/w % of a preservative; and wherein said vehicle contains less than 0.5 w/w % of fragrance and less than 0.5 w/w % of a performance agent.

2. The composition of claim 1 wherein the relative percentage increase in viscosity of said personal care product doesn't exceed about 3% after the addition of said vehicle in substitution for at least one component selected from said fragrance and said performance agent.

3. The composition of claim 1 wherein the relative percentage increase in viscosity of said personal care product doesn't exceed about 1% after the addition of said vehicle in substitution for at least one component selected from said fragrance and said performance agent.

4. The composition of claim 1, wherein said solvent is present in an amount of at least about 90 w/w %; said at least one solubilizing agent is present in a total amount below about 10 w/w %, said solubilizing agents being selected from a polyethylene glycol ether of a fatty alcohol, a polyethylene glycol ether of hydrogenated castor oil, a polyethylene glycol derivative of a sorbitan ester, a polysorbate, propylene glycol, a glycerol ester, a-polyethylene glycol derivative of a glycerol ester, an alkyl phosphate, and an alkyl sulfate; said preservative is present in an amount below about 5.0 w/w %; and there are substantially no fragrance or performance agents present.

5. The composition of claim 1 wherein said solvent is selected from water, a monohydric alcohol, a polyhydric alcohol, or a blend thereof.

6. The composition of claim 1 wherein said preservatives are selected from DMDM Hydantoin, lodopropynyl Butylcarbamate, polyaminocarboxylic acid chelates and salts thereof, and phosphonate chelates and salts thereof.

7. The composition of claim 1 which whose performance properties remain stable after being blended with at least one fragrance concentrate in a concentration of about 0.01 to 10.0 w/w %; and with at least one personal care composition base in a concentration of about 10.0 to 95.0 w/w %.

8. The composition of claim 7 wherein said fragrance is present in a concentration of about 0.05 to 5.0 w/w %.

9. The composition of claim 7 whose performance properties remain stable after being blended with at least one benefit agent in a concentration of about 0.01 to 10.0 w/w %.

10. The composition of claim 9 wherein said benefit agent is present in a concentration of 0.1 to 5.0 w/w %.

11. The composition of claim 9, wherein said benefit agent is selected from botanical extracts, vegetable oils, vitamins, protein, honey, or blends thereof.

12. The composition of claim 9, wherein said personal care composition base is selected from a body wash, a body lotion, a body mist spray, a hydroalcoholic toner, a facial cleansing gel, a hand cleanser, a hair shampoo, a hair conditioner, a face lotion, a deodorant, a bar soap, a bath foam, and bath salts.

13. A fragrance concentrate, comprising:
from about 5 to about 80 w/w % of a fragrance composition;
from about 0.001 to about 30 w/w % of a colorant;
from about 5 to about 80 w/w % of at least one solvent; and
from about 0.01 to 10 w/w % of a preservative.

14. The fragrance concentrate of claim 13 wherein said fragrance composition is present from about 40 to about 60 w/w %; said colorant is present from about 0.01 to about 0.5 w/w %; said solvent is present from about 40 to about 60 w/w %; and said preservative is present from about 0.01 to 5 w/w %.

15. The fragrance concentrate of claim 13, wherein the solvent is selected from water, a monohydric alcohol, a polyhydric alcohol, or a blend thereof.

16. The fragrance concentrate of claim 13, wherein the preservative is selected from DMDM Hydantoin, lodopropynyl Butylcarbamate, polyaminocarboxylic acid chelates and salts thereof, and phosphonate chelates and salts thereof.

17. The fragrance concentrate of claim 13 which further contains a color-stabilizing agent.

18. The fragrance concentrate of claim 15 wherein the color stabilizer is selected from benzophenone-2, benzophenone-3, benzophenone-4, octylmethoxycinnamate, and urocanic acid
